SEQUENCE LISTING (11 sequences)

SEQ ID NO: 1 - DNA, 4332 bp, Artificial Sequence
  Description of Artificial SequencepMB1-MnSOD plasmid expression vector
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgataagcttgatatcgaattccgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactggggagaatgtaactgaaagatacatggcttgcaaaaagtaaaccacgatcgttatgctggaaaaaaaaaaaaaaaaaaaaaaaaaaaaaaaaaaaaaaaaaaaaaaanaaaaaaacggaattccctgcagcccgggggatccactagttctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcag

SEQ ID NO: 2 - DNA, 4293 bp, Artificial Sequence
  Description of Artificial SequencepMB1-HAMnSOD plasmid expression vector
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgataagcttgataattccaccatggcttctagcccttatgacgtgcctgactatgccagtttaggaggaccttctatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactggggagaatgtaactgaaagatacatggcttgcaaaaagtaaaccacgatcgttatgctggaaaaaaaaaaaaaaaaaaaaaaaaaaaaaaaaaaaaaaaaaaaaaaanaaaaaaacggaattccctgcagcccgggggatccactagttctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcag

SEQ ID NO: 3 - DNA, 5753 bp, Artificial Sequence
  Description of Artificial SequencepMB1-CAT plasmid expression vector
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgataagcttgatatcgaattcgggtggagacccacgagccgaggcctcctgcagtgttctgcacagcaaaccgcacgctatggctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctcgagtggccaactaccagcgtgatggcccgatgtgcatgcaggacaatcagggtggtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctgtgaggccggggccctgcacctgtgcagcgaagcttagcgttcatccgtgtaacccgctcatcactggatgaagattctcctgtgctagatgtgcaaatgcaagctagtggcttcaaaatagagaatcccactttctatagcagattgtgtaacaattttaatgctatttccccaggggaaaatgaaggttaggatttaacagtcatttaaaaaaaaaatttgttttgacggatgattggattattcatttaaaatgattagaaggcaagtttctagctagaaatatgattttatttgacaaaatttgttgaaattatgtatgtttacatatcacctcatggcctattatattaaaatatggctataaatatataaaaagaaaagataaagatgatctactcagaaatttttatttttctaaggttctcataggaaaagtacatttaatacagcagtgtcatcagaagataacttgagcaccgtcatggcttaatgtttattcctgataataattgatcaaattcatttttttcactggagttacattaatgttaattcagcactgatttcacaacagatcaatttgtaattgcttacatttttacaataaataatctgtacggaattcctgcagcccgggggatccactagttctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcag

SEQ ID NO: 4 - DNA, 5760 bp, Artificial Sequence
  Description of Artificial SequencepMB1-Mt-CAT plasmid expression vector
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctagtaacggccgccagtgtgctggaattcgcccttccccaccatgttgagccgggcagtgtgcggcaccagcaggcagctggctccggctttggggtatctgggctccaggcagatggctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctcgagtggccaactaccagcgtgatggcccgatgtgcatgcaggacaatcagggtggtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctgtgaggccggggccctgcacctgtgcagcgaagcttagcgttcatccgtgtaacccgctcatcactggatgaagattctcctgtgctagatgtgcaaatgcaagctagtggcttcaaaatagagaatcccactttctatagcagattgtgtaacaattttaatgctatttccccaggggaaaatgaaggttaggatttaacagtcatttaaaaaaaaaatttgttttgacggatgattggattattcatttaaaatgattagaaggcaagtttctagctagaaatatgattttatttgacaaaatttgttgaaattatgtatgtttacatatcacctcatggcctattatattaaaatatggctataaatatataaaaagaaaagataaagatgatctactcagaaatttttatttttctaaggttctcataggaaaagtacatttaatacagcagtgtcatcagaagataacttgagcaccgtcatggcttaatgtttattcctgataataattgatcaaattcatttttttcactggagttacattaatgttaattcagcactgatttcacaacagatcaatttgtaattgcttacatttttacaataaataatctgtacggaattcctgcagcccgggggatccactagttctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcag

SEQ ID NO: 5 - DNA, 4058 bp, Artificial Sequence
  Description of Artificial Sequence pMB1-hIFN-alpha plasmid expression vector
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgataagcttgatatcacagaggagaccatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgagctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcag

SEQ ID NO: 6 - DNA, 4187 bp, Artificial Sequence
  Description of Artificial SequencepMB1-EcSOD plasmid expression vector
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggccaccatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcacgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcgcaagaagcggcggcgcgagagcgagtgcaaggccgcctgaaagcttgatatcgaattcctgcagcccgggggatccactagttctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcag

SEQ ID NO: 7 - DNA, 6326 bp, Artificial Sequence
  Description of Artificial SequencepBAT-R1-CAT plasmid expression vector
aatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgataagcttgatatcgaattcgggtggagacccacgagccgaggcctcctgcagtgttctgcacagcaaaccgcacgctatggctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctcgagtggccaactaccagcgtgatggcccgatgtgcatgcaggacaatcagggtggtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctgtgaggccggggccctgcacctgtgcagcgaagcttagcgttcatccgtgtaacccgctcatcactggatgaagattctcctgtgctagatgtgcaaatgcaagctagtggcttcaaaatagagaatcccactttctatagcagattgtgtaacaattttaatgctatttccccaggggaaaatgaaggttaggatttaacagtcatttaaaaaaaaaatttgttttgacggatgattggattattcatttaaaatgattagaaggcaagtttctagctagaaatatgattttatttgacaaaatttgttgaaattatgtatgtttacatatcacctcatggcctattatattaaaatatggctataaatatataaaaagaaaagataaagatgatctactcagaaatttttatttttctaaggttctcataggaaaagtacatttaatacagcagtgtcatcagaagataacttgagcaccgtcatggcttaatgtttattcctgataataattgatcaaattcatttttttcactggagttacattaatgttaattcagcactgatttcacaacagatcaatttgtaattgcttacatttttacaataaataatctgtacggaattcctgcagcccgggggatccactagttctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 8 - DNA, 6331 bp, Artificial Sequence
  Description of Artificial SequencepBAT-PCR-CAT plasmid expression vector
aatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctccaccatggctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctcgagtggccaactaccagcgtgatggcccgatgtgcatgcaggacaatcagggtggtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctgtgaggccggggccctgcacctgtgcagcgaagcttagcgttcatccgtgtaacccgctcatcactggatgaagattctcctgtgctagatgtgcaaatgcaagctagtggcttcaaaatagagaatcccactttctatagcagattgtgtaacaattttaatgctatttccccaggggaaaatgaaggttaggatttaacagtcatttaaaaaaaaaatttgttttgacggatgattggattattcatttaaaatgattagaaggcaagtttctagctagaaatatgattttatttgacaaaatttgttgaaattatgtatgtttacatatcacctcatggcctattatattaaaatatggctataaatatataaaaagaaaagataaagatgatctactcagaaatttttatttttctaaggttctcataggaaaagtacatttaatacagcagtgtcatcagaagataacttgagcaccgtcatggcttaatgtttattcctgataataattgatcaaattcatttttttcactggagttacattaatgttaattcagcactgatttcacaacagatcaatttgtaattgcttacatttttacaataaataatctgtacgtaagaacagaaaaaaaaggaattccgtgtattctatagtgtcacctaaatcgtatgtgagatctctcgaggtcgacggtatcgataagcttgatatcgaattcctgcagcccgggggatccactagttctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 9 - DNA, 21 bp, Artificial Sequence
  Description of Artificial Sequenceprimer specific for 5′UTR of pBAT/pMB1 plasmid vector
ggagacgccatccacgctgtt

SEQ ID NO: 10 - DNA, 20 bp, Artificial Sequence
  Description of Artificial Sequence3′ oligo specific for human MnSOD gene
gcgcgttgatgtgaggttcc

SEQ ID NO: 11 - DNA, 22 bp, Artificial Sequence
  Description of Artificial Sequencecatalase specific primer
cgataccgtcgacctcgagaga

(19) United States
(12) Patent Application Publication
(10) Pub. No.: US 2005/0043258 A1
(43) Pub. Date: Feb. 24, 2005

(54) Methods of treating xerostomia and xerophthalmia

(75) Inventors: Michael Bennett, El Sobrante, CA (US); Yen-Ju Chen, Alameda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Genteric, Inc., Alameda, CA (US)

(21) Appl. No.: 10/811,028

(22) Filed: Mar. 25, 2004

Related U.S. Application Data
(60) Provisional application No. 60/458,793, filed on Mar. 26, 2003.

(51) Int. Cl. A61K 48/00

(52) U.S. Cl. 514/44

(57) ABSTRACT

The present invention provides methods for protecting or treating a tissue from a condition that elicits xerostomia or xerophthalmia.

### BACKGROUND OF THE INVENTION

The present application claims priority to U.S. Provisional Patent Application No. 60/458,793, filed Mar. 26, 2003, the disclosures of which are hereby incorporated by reference in their entirety for all purposes.

### BACKGROUND OF THE INVENTION

Xerostomia (dry mouth) results from damage to salivary gland acinar cells after radiation treatment for head and neck cancer, and as a result of various autoimmune disorders, such as Sjögren's syndrome. The major salivary glands—parotid, submandibular and sublingual—are comprised of secretory cells and ductal cells. The secretory cells are largely responsible for the fluid in the mouth. Oral symptoms of xerostomia include: dry mouth, difficulty eating, an increase in the prevalence of dental caries, recurrent yeast infection, retention of food and bacteria, and swelling and tenderness of the major salivary glands. Xerostomia is often irreversible, and most patients never return to normal salivary secretion levels. Most current treatments for xerostomia are palliative in nature.

The current standard treatment for Xerostomia is amifostine (Ethyoll), which is infused immediately preceding radiation treatment. Amifostine reduces the incidence of moderate to severe xerostomia by thirty-five percent. However, there are several disadvantages to this mode of therapy: 1) greater than fifty percent of patients that receive amifostine develop moderate to severe xerostomia, 2) serious side effects are observed in seventeen percent of patients, and 3) side effects include: nausea, vomiting, hypotension, fever, allergic-type skin reactions, dizziness, lethargy, chills, sleepiness, and flushing. In addition, amifostine requires frequent administration and has limited effectiveness.

Prior to irradiation, viral or non-viral transfer of the MnSOD gene to the mouse lung has been shown to protect the lungs from irradiation damage (see, U.S. Pat. No. 5,599,712 and Epperly et al. 1998, Gene Ther. 5:196-208). Baum et al. have theorized on, and reported efforts to use gene therapy for restoration of salivary gland function after cancer therapy or autoimmune disease (see, Baum et al. 2002, International Review of Cytology, 213:93-146). This involves in vivo engineering of the surviving duct cells, which cannot produce saliva, to express genes that encode proteins such as aquaporin. These proteins can allow water to flow into the lumen of the duct, in theory partially ameliorating the dry mouth syndrome. These experiments used adenovirus to deliver the aquaporin gene to irradiate rats and showed that salivary hypofunction can be corrected. To date, these efforts have been limited to animal experiments.

There are 40,000 new cases of head and neck cancer diagnosed per year in the United States. A need therefore exists for an effective therapy or palliative for dry mouth syndrome resulting from salivary gland destruction after radiotherapy or chemotherapy and as a result of various autoimmune disorders, such as Sjögren's syndrome. The present invention meets this and other needs by providing pretreatment options including transient application to salivary gland cells of genes, proteins or chemicals that provide anti-oxidant or radioprotection. In addition, the present invention describes the use of catheters to apply fluid that contains genes, protein, and/or chemicals that are radioprotective in the ductal space.

### BRIEF SUMMARY OF THE INVENTION

The present invention provides methods and compositions for treating xerostomia and xerophthalmia in animals, including humans. In one embodiment of the invention, a method is provided for attenuating increases in the concentrations of harmful agents including radiation-induced free radicals, superoxide anions and heavy metal cations in a mammalian cell comprising the steps of contacting a cell with one or more nucleic acids encoding one or more proteins, wherein one or more of the proteins, when expressed, neutralizes or eliminates a portion of the free radicals, superoxide anions or heavy metal cations in the targeted cell. In a preferred embodiment, multiple cells are targeted and the proteins delivered to the targeted cells are expressed in a plurality of cells. In a preferred embodiment, the targeted cell is a mammalian cell. In another preferred embodiment, the targeted cell is a human cell.

In a preferred embodiment, the beneficial encoded protein(s) delivered to the cell may include a metallothionein, superoxide dismutase, catalase, glutathione peroxidase (GPx)-4, or gamma glutamyl transpeptidase. In one embodiment, the vector used for delivery encodes only one of the aforementioned proteins. In a related embodiment, a combination of the aforementioned proteins is delivered simultaneously by one or more vectors. In yet another embodiment, the proteins are delivered to the targeted cells at different times, as needed.

In one embodiment, the invention provides a method for delivery of a vector encoding a superoxide dismutase to a cell where the superoxide dismutase is either copper-zinc superoxide dismutase, manganous superoxide dismutase or iron superoxide dismutase.

In a preferred embodiment, the transfection method provided for attenuating increases in the cellular concentration of free radicals, superoxide anions or heavy metal cations is practiced prior to the irradiation of the cell. In a related embodiment, the transfection method provided for attenuating increases in the cellular concentration of free radicals, superoxide anions or heavy metal cations is practiced after the irradiation of the cell. The irradiation of the cell is typically irradiation for the treatment of cancer.

In another embodiment, the invention provides a method for contacting a salivary gland cell in an animal with a nucleic acid encoding a protein capable of attenuating cellular levels of free radicals, superoxide anions or heavy metal cations, wherein the contacting is repeated on a plurality of cells, and wherein expression of the encoded proteins is sufficient to amelerioate a symptom of xerostomia in the animal.

In an embodiment related to the embodiments described above, the invention provides a method for contacting a lacrimal gland cell in an animal with a nucleic acid encoding a protein capable of attenuating cellular levels of free radicals, superoxide anions or heavy metal cations, wherein the contacting is repeated on a plurality of cells, and wherein expression of the encoded proteins is sufficient to ameliorate a symptom of xerophthalmia in the animal.

In certain embodiments of the methods of the invention described above, the nucleic acid which is delivered to the targeted cells is an expression vector. In a preferred embodiment, the expression vector is selected from the group of expression vectors described in FIG. 1A-H, i.e., the vectors of SEQ ID NOs 1-8.

In another preferred embodiment, the composition delivered to the targeted cells for attenuating cellular levels of free radicals, superoxide anions or heavy metal cations includes a polyionic organic acid. In a related embodiment, the polyionic organic acid composition includes a transition metal enhancer, for example, zinc.

The invention also provides nucleic acid compositions encoding a protein or peptide which may be used, for example, in the methods of the invention. In one embodiment, the nucleic acid is a DNA, a RNA, a DNA/RNA hybrid, an antisense oligonucleotide, a chimeric DNA-RNA polymer, a ribozyme, or a plasmid DNA. In one embodiment, the protein remains in the secretory gland. In another embodiment, the protein is gamma glutamyl transpeptidase, manganese superoxide dismutase, metallothionein, glutathione peroxidase (GPx)-4 or catalase. In another aspect, the protein is IFN-alpha, IL-10, sTNFR, TGF-β, IL-4, VIP, anti-TNF antibody, IL1-RA, other antibodies to proinflammatory cytokines, soluble gp39, soluble CD40, aquaporin-1 and aquaporin-5. In another aspect, the composition further comprises an ionizable or ionized transition metal enhancer. In a preferred embodiment of the compositions of the invention, the encoded proteins include gamma glutamyl transpeptidase, manganese superoxide dismutase, or metallothionein. In another preferred embodiment, the invention provides the vectors described in FIG. 1A-H and SEQ ID Nos. 1-8. In certain aspects, the composition is delivered to the mammal by intraductal delivery or direct administration, electroporation administration, or ultrasound administration.

In another aspect, the subject to be treated is undergoing, has undergone, or is about to undergo radiation treatment for cancer. In another aspect, the condition being treated by the methods and compositions of the method is caused by a harmful species such as a free radical, a super oxide anion, or a heavy metal cation. In another embodiment, the condition to be treated is ionizing radiation, an autoimmune disorder, Sjögren's syndrome, graft-versus-host disease, systemic lupus erythematosis, rheumatoid arthritis, HIV-1 infection, ageing, treatment with medications/drugs, autonomic dysfunction, conditions affecting the CNS, psychogenic disorder, trauma, or decrease in mastication.

In another embodiment, the present invention provides a method for protecting or treating a tissue from a condition that elicits xerostomia or xerophthalmia, comprising: retroductally introducing into the lumen of a salivary gland duct of a subject a composition to neutralize a harmful species, thereby protecting or treating said tissue from a condition that elicits xerostomia or xerophthalmia. In one aspect, the tissue is a secretory gland. In one embodiment, the secretory gland is a salivary gland, a pancreas, a mammary gland, a thyroid, a thymus, a pituitary gland, a lacrimal gland, or a liver. In another embodiment, the tissue is the oral cavity, oropharynx, esophagus, small intestine or colon.

In another embodiment, the invention provides a method for method for ameliorating a symptom of xerostomia in a mammal comprising the steps of contacting a salivary gland cell of said mammal with a composition comprising a protein selected from the group consisting of IFN-alpha, IL-10, sTNFR, TGF-β, IL-4 and VIP, anti-TNF antibody, IL1-RA, other antibodies to proinflammatory cytokines, soluble gp39, soluble CD40, aquaporin-1 and aquaporin-5. In a preferred embodiment, the protein is IFN-alpha. In a related embodiment, the mammal is a human and the symptom of xerostomia is associated with a condition such as an autoimmune disorder, Sjogren's syndrome, graft-versus-host disease, systemic lupus erythematosis, rheumatoid arthritis, HIV-1 infection, ageing, autonomic dysfunction, conditions affecting the CNS, psychogenic disorder, trauma, hepatitis C, cancer, decrease in mastication or a combination thereof. In a related embodiment, at least a portion of the encoded protein is expressed by salivary gland cells and secreted systemically into blood plasma or into the oral cavity. In a preferred embodiment, the secreted protein is IFN-alpha. In yet another embodiment, the composition for contacting the targeted cells with a nucleic acid encoding IFN-alpha further comprises a polyionic organic acid and, even more preferably, additionally includes a transition metal enhancer.

These and other aspects of the invention are described in more detail in the sections which follow.

### DESCRIPTION OF THE FIGURES

FIG. 1A-H shows maps of the various vectors used in the Examples presented herein. The corresponding SEQ ID Nos for the vectors are as follows: SEQ ID 1, pMB1-MnSOD; SEQ ID 2, pMB1-HAMnSOD; SEQ ID 3, pMB 1-CAT; SEQ ID 4, pMB1-Mt-CAT; SEQ ID 5, pMb1-hIFN-alpha; SEQ ID 6, pMB1-EcSOD; SEQ ID 7, pBAT-R1-CAT; and SEQ ID 8, pBAT-PCR-CAT. FIG. 1 includes the following abbreviations: KAN, kanamycin resistance gene; CMV, human cytomegalovirus IE1 promoter region (Accession No. X03922); OR1, ColE1 bacterial origin of replication from pBluescript SK+; AMP, ampicillin resistance gene (beta-lactamase); HBG intron, human beta globin intron; ECSOD, extracellular superoxide dismutase; MnSOD, manganese superoxide dismutase; hIFN-alpha, human alpha interferon; HA-MnSOD, recombinant fusion protein including a hemaglutinin epitope tag. “Mito leader” refers to the presence of a mitochondrial leader sequence. Bgl II, NotI, and AvrI denote restriction sites for the indicated restriction enzymes. FIG. 11 is a table which provides additional details relating to the vector constructs.

FIG. 2 shows the detection of recombinant catalase transcription in transfected rat submandibular glands using RT-PCR. Abbreviations: RT, reverse transcription PCR product corresponding to transcribed catalase; L and R refer to tissue samples taken from the left and right submandibular glands of the transfected animal.

FIG. 3 shows a comparison of catalase expression in rat submandibular glands transfected with two different catalase vectors, pBATR1-CAT and pBAT-PCR-CAT versus (1) glands transfected with a control vector (pSEAP) and (2) untreated “naïve” glands. The number of different animals transfected and analyzed for each vector is given as “n.” Errors bars correspond to the standard error of the mean (“SEM”).

FIG. 4 shows the detection of recombinant MnSOD transcription in transfected rat submandibular glands using RT-PCR. Abbreviations: L and R refer to tissue samples taken from the left and right submandibular glands of the transfected animal; KB, molecular weight size markers.

FIG. 5 shows the expression of recombinant MnSOD protein in vivo in transfected rat submandibular glands. Recombinantly expressed MnSOD and HA-MnSOD were visualized using antibody staining of thinly sliced tissue sections.

FIG. 6 shows that human interferon alpha protein is systemically expressed in the blood plasma of rats whose submandibular glands were transfected with a vector (pMB1-hIFN-alpha) encoding human interferon alpha. Abbreviations: pg, picograms.

### DETAILED DESCRIPTION OF THE INVENTION

I. Definitions

As used herein, the following terms have the meanings ascribed to them below unless otherwise specified.

“Xerostomia,” or “dry mouth,” is a symptom associated with a decrease in salivary flow and/or alterations in salivary composition. Xerostomia is manifested in complaints of oral dryness, burning of the tissues, difficulty eating and swallowing, irritation of the tongue and painful ulcerations as well as significantly progressive caries and periodontal disease. The burning tongue (glossodynia) associated with xerostomia may become quite severe with chronic dryness, resulting in atrophy and painful fissuring and desquamation of the mucosa, often interfering with nutritional intake. Oral symptoms of xerostomia include: dry mouth, difficulty eating, increase in the prevalence of dental caries, recurrent yeast infection, retention of food and bacteria, and swelling and tenderness of the major salivary glands. Most current treatments for xerostomia are palliative in nature.

“Xerophthalmia” is excessive drying of the conjunctiva and cornea and may be due to a local disease, insult, or a vitamin A deficiency. Xerosis and excessive drying of the cornea may be seen initially as a mild haziness inferiorly. This is followed by corneal ulceration with full thickness dissolution known as keratomalacia and possible extrusion of the intraocular contents. Left untreated, xerophthalmia can lead to night blindness.

“Sjögren's syndrome” is a chronic, autoimmune rheumatic disorder characterized by the destruction of exocrine glands by lymphocytes in association with autoantibody production or as a complication of a previously existing connective tissue disorder. Over time, lacrimal and salivary glands are infiltrated by mononuclear cells which leads to decreased secretions, with resultant xerostomia (dry mouth) and xerophthalmia (dry eyes) being the most prevalent symptoms.

A “salivary gland” is a gland of the oral cavity which secretes saliva, including the glandulae salivariae majores of the oral cavity (the parotid, sublingual, and submandibular glands) and the glandulae salivariae minores of the tongue, lips, cheeks, and palate (labial, buccal, molar, palatine, lingual, and anterior lingual glands).

“Retroductally introducing” refers to introduction of a composition through a duct in a salivary gland, wherein the composition flows through the salivary gland duct in a retrograde manner. Suitable ducts include all major and minor salivary gland ducts. For example, the Wharton's duct or the Stenson's duct is suitable.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleotides may be referred to by their commonly accepted single-letter codes. These are A, adenine; C, cytosine; G, guanine; and T, thymine (DNA), or U, uracil (RNA).

The term “codon” refers to a sequence of nucleotide bases that specifies an amino acid or represents a signal to initiate or stop a function. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605 (1985); Rossolini et al., Mol. Cell. Probes 8:91 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to naturally occurring amino acid polymers, as well as, amino acid polymers in which one or more amino acid residue is an artificial chemical mimetic of a corresponding naturally occurring amino acid.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified through posttranslational modification, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Conservatively modified variants” applies to both nucleic acid and amino acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

Each of the following eight groups contains amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The term “contacting,” in the context of “contacting cells” or “contacting a tissue” with a nucleic acid vector or gene, refers to a process whereby a vector or gene is delivered to the interior of a cell or cells of interest or placed in such proximity to the cell or cells of interest that some portion of the vector or gene passes through the cell membrane(s) and into the interior of the cell(s) where any proteins (e.g., MnSOD, CuSOD, catalase, IFN-alpha, etc.) or nucleic acids (e.g., tRNA, RNAs, siRNAs, and the like) encoded by the gene are subsequently expressed. The vector may be delivered to the cell or tissue directly (e.g., by injection at the cell or tissue site) or indirectly (e.g., injection into the bloodstream, following administration of the vector at a site remote from the tissue to be contacted).

A nucleic acid administered to the salivary gland may be encapsulated in a liposome (or other cationic, anionic, or neutral polymer) formulation.

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

“Transition metal enhancer” as used herein refers to compounds having one or more transition metal atoms selected from the elements in Groups IIIB, IVB, VB, VIIB, VIIIB, IB, and IIB of the periodic table (i.e., the d-block) (see, e.g., Huheey, INORGANIC CHEMISTRY, Harper & Row, New York, 1983). The transition metals of the present invention also include those lanthanides (i.e., the first row of thef-block of the periodic table) and main group metals (i.e., groups IIIA, IVA, VA, and VIIA of the periodic table), having chemical properties similar to transition metal complexes.

A nucleic acid may be administered to a gland, e.g., a salivary or lacrimal gland, with or without a “formulant,” i.e., a substance that enhances transfection efficiency. Suitable formulants include, for example, divalent transition metals and polyanionic organic acids. “Divalent transitions metal compounds” refer to compounds comprising a divalent transition metal, such as, for example, zinc, copper, cobalt, or nickel.

A “polyionic organic acid” (POD) as used herein, is preferably a polyprotic polyaromatic organic compound wherein the compound contains at least two aromatic components. “Polyionic” compounds refer to compounds comprising one or more ionizable units, either as in the protonated form or as the conjugate salt. In certain embodiments, the POD has associated therewith, such as complexed with, a transition metal enhancer of the type described below. In certain other embodiments, the POD acts directly as an antiviral compound.

The term “in vivo” refers to being within a living organism such as a plant or animal, and includes, but is not limited to, cells, tissues, glands, organs, and the like, in the living organism. The term “in vitro” refers to an artificial environment outside a living organism, and includes, but is not limited to, cells, tissues, glands, organs, and the like, outside of the living organism.

A “therapeutic protein” or “therapeutic nucleic acid” is any protein or nucleic acid that provides a therapeutic or prophylactic effect. A therapeutic protein may be naturally occurring or produced by recombinant means. A “therapeutically effective amount” of a nucleic acid or protein is an amount of nucleic acid or protein sufficient to provide a therapeutic or prophylactic effect in a subject. Such therapeutic or prophylactic effects may be local or systemic. Therapeutic and prophylactic effects include, for example, eliciting or modulating an immune response. Selby et al. (2000) J Biotechnol. 83(1-2):147-52. Immune responses include humoral immune responses and cell-mediated immune responses.

“Electroporation” involves contacting cells, tissues, glands, or organs with electrodes and “pulsing” the cells, tissues, glands, or organs, i.e., passing an electric signal through the tissues, glands, or organs via the electrode. One preferred embodiment of the present invention comprises contacting a salivary gland with an electrode and “pulsing” the salivary gland. “Contacting,” in this context, includes placing the electrodes at or near the cells, tissues, glands, or organs; touching the cells, tissues, glands, or organs with the electrodes, or penetrating the tissues, glands or organs with the electrodes. After contacting and pulsing the salivary gland, electrodes may be “repositioned” to come into contact with the same or different position on the salivary gland. After repositioning of the electrode, the salivary gland may be pulsed again. “Electrodes” that can be used to contact the cells, tissues, glands, or organs, include needles, laparoscopic needles, probes, needles with paddles, and needles with flat plates or calipers. Electrodes may comprise individual needles, laparoscopic needles, probes, needles with paddles, and flat plates or may comprise an array of multiple needles, e.g., 2, 4, 6, 8, 10, 12, 14, 16, 18, 20, 22, 24, 26, 28, or 30 needles, laparoscopic needles, probes, needles with paddles, and needles with flat plates or calipers.

II. Methods for Treating Xerostomia and Xerophthalmia

The present invention provides in part, methods for treating symptoms associated with a decrease in salivary flow and/or alterations in salivary composition. In one aspect, the present invention provides a method for protecting or treating a tissue from a condition that elicits xerostomia or xerophthalmia comprising administering to a subject a composition comprising a nucleic acid encoding a protein capable of neutralizing a harmful species. In certain aspects, the harmful species can include free radicals, superoxide anions, and heavy metal cations. The present invention is also directed to prophylactically protecting an individual's tissues against the damaging effects of a harmful species, e.g., a free radical, superoxide anion or heavy metal cation resulting from ionizing radiation used in cancer therapy, prior to exposure to the harmful species.

Conditions that elicit xerostomia or xerophthalmia include, for example, ionizing radiation, an autoimmune disorder, Sjögren's syndrome, graft-versus-host-disease, systemic lupus erythematosis, rheumatoid arthritis, HIV-1 infection, ageing, treatment with medications/drugs, autonomic dysfunction, conditions affecting the CNS, psychogenic disorder, trauma, decrease in mastication, or a combination thereof.

In a preferred embodiment, the methods of the present invention protect against ionizing radiation which produces a free-radical species. For example, the use of ionizing radiation to treat head and neck tumors causes the formation of superoxide ions (O²⁻) and hydroxyl radicals (OH.):
O₂⁻.+H₂O₂=>OH.+OH⁻
These reactive oxygen species (ROS) lead to the peroxidation of lipids and the oxidation of most organic molecules. Strategies directed towards reducing superoxide and hydrogen peroxide concentrations have been effective at reducing oxidative damage to tissues in small animal models.

Antineoplastic agents, particularly the class of chemotherapeutic drugs known as alkylating agents, also produce free radicals that are cytotoxic because of their ability to form covalent bonds with nucleic acids. Most alkylating agents, including cyclophosphamide, nitrogen mustard, melphalan, chlorambucil, busulfan, nitrosourea, cis-platinum, streptozotocin, aziridinylbenzoquinone (AZQ), dicarbazine (DTIC), mAMSA and mitoxantrone, form positively charged carbonium ions that yield a charged alkylating intermediate that attacks electron-rich sites on nucleic acids, proteins, small molecules and amino acids.

In certain aspects, the methods of the present invention provide a means for protecting or treating cells, particularly a secretory gland cell such as a salivary gland cell, a pancreatic cell, a mammary gland cell, a thyroid cell, a thymus cell, a pituitary gland cell, a lacrimal cell and a liver cell. Further cells include, for example, the cells of the oral cavity including, but not limited to, oropharynx, esophagus, stomach, small intestine and colon. In certain aspects, the methods protect an individual against a harmful species that elicits the production of a free radical, a superoxide anion, and/or a heavy metal cation. The cells to be protected may be located at a site remote from the site of delivery of the recombinant nucleic acid or protein. In other embodiments, the protein delivered remains in the secretory gland. In other embodiments, the protein delivered is secreted locally within the gland. In still other embodiments, the protein delivered is secreted systemically.

In another embodiment, the present invention provides a method for protecting or treating a tissue from a condition that elicits xerostomia or xerophthalmia, comprising: retroductally introducing into the lumen of a salivary gland duct of a subject a composition to neutralize a harmful species, thereby protecting or treating the tissue from a condition that elicits xerostomia or xerophthalmia. Preferably, the composition includes a nucleic acid encoding a protein which is capable of neutralize a harmful species, e.g., a free radical, resulting from ionizing radiation.

A. Recombinant Proteins Useful for Protecting Subjects from Symptoms of Xerostomia and Xeropthalmia

In certain aspects, the compositions useful in the practice of the present invention comprise a protein or a nucleic acid encoding a protein. In other aspects, the composition comprises a protein, a polyionic organic acid, a nucleic acid encoding a protein or a combination thereof. In still other embodiments, the compositions comprise nucleic acids with intrinsic activities of their own, e.g., tRNAs, siRNAs, and antisense RNAs. Additional nucleic acids useful in the present invention include, for example, DNA, RNA, DNA/RNA hybrids, antisense oligonucleotides, chimeric DNA-RNA polymers, ribozymes, plasmid DNAs, nucleic acids encoding proteins, therapeutic proteins, antibodies, peptides, cyclic peptides and ribozymes.

In certain aspects, the protein is selected from the group of gamma glutamyl transpeptidase, manganese superoxide dismutase, metallothionein, glutathione peroxidase (GPx)-4 and catalase.

In certain preferred aspects, the methods of the present invention provide an in vivo method for delivering to a cell of interest a polynucleotide encoding a protein capable of neutralizing or eliminating a toxic free radical, superoxide anion and/or heavy metal cation, wherein the protein is transiently expressed in the individual. The transgenes of the present invention encode proteins such as metallothionein, superoxide dismutase or gamma glutamyl transpeptidase, that scavenge heavy metal cations, superoxide anions and/or toxic free radicals.

In one embodiment, the invention provides compositions and methods for the delivery of glutamyltranspeptidease (γ-GTP) to cells. γ-GTP is a plasma membrane-associated ectoenzyme that catalyzes the transpeptidation of extracellular glutathione into amino acid intermediates, which are then transported across the cell membrane and used to resynthesize glutathione de novo. Glutathione (GSH) detoxifies free-radicals. Cells generally synthesize GSH de novo from the constituent amino acids. A cell's sensitivity to radiation is directly correlated with its ability to transpeptidate extracellular glutathione via γ-GTP. Cell lines with high γ-GTP activity are more resistant to the effects of radiation and are more capable of repairing damage induced by low doses of γ-irradiation than cell lines with low Y-GTP activity.

In a related embodiment, the invention provides compositions and methods for the delivery and expression of proteins which protect against superoxide radicals. Such proteins include antioxidants such as GSH, described above, and the radical scavenging enzyme superoxide dismutase (SOD). At least three forms of SODs have been identified: copper-zinc (CuZnSOD), manganous (MnSOD) and iron (FeSOD). Although CuZnSOD and FeSOD are made constituitively, MnSOD synthesis is inducible.

MnSOD has been shown to prevent irradiation damage to tissues, and also protects against radiation-induced esophagitis and mucocitis. In addition, non-viral MnSOD gene transfer is effective in protecting tissues from irradiation damage.

Endogenous proteins such as MnSOD (mitochondrial), Cu/ZnSOD (cytosolic), and ECSOD (extracellular) are effective at reducing superoxide concentrations by catalyzing the following reaction:
O₂⁻.+O₂⁻.+H₂=>H₂O₂+O₂

In a further embodiment, the invention provides compositions and methods for the delivery and expression of catalase in a cell. Proteins such as catalase and glutathione peroxidase are effective at reducing hydrogen peroxide concentrations by catalyzing the following reaction:
2H₂O₂=>2H₂O+O₂
Additional relevant activities and properties of catalase are discussed, for example, by Mao et al., (J. Biol. Chem., 258:416-420 (1992)); Bai et al. (J. Biol. Chem., 274:26217-26224 (1999)).

In another embodiment, the invention provides compositions and methods for the delivery and expression of a metallothionein. Metallothionein protein protects cells from the toxic effects of heavy metal ions and is a powerful scavenger of radiation-induced OH-radicals in vitro. Human metallothionein, for example, is a low molecular weight protein consisting of a single polypeptide chain of 61 amino acid residues, of which 20 are cysteines that chelate cations. Induction of metallothionein has been shown to provide resistance to ionizing irradiation damage. Cells lines that express high levels of MT are resistant to DNA damaging agents, such as cis-platinum and chlorambucil, and ionizing radiation. Additional relevant activities of metallothionein are discussed, for example, by Manuel et al. (IARC Sci Publ., 118:231-7 (1992)); Coyle et al. (Cell Mol Life Sci., 59:627-47 (2002)).

In a further embodiment, the invention provides compositions and methods for the delivery and expression of IFN-alpha to cells, particularly to salivary gland cells and lacrimal gland cells. IFN-alpha has the capacity to inhibit tumor cell proliferation via antagonistic effects on growth factors and is an essential cofactor for the generation of a systemic immune response following prodrug-induced tumor cell killing. IFN-alpha is also believed to upregulate aquaporin-5 gene expression.

In yet another embodiment of the invention, any of the proteins described above, including catalase, IFN-alpha, glutathione peroxidase, glutamyltranspeptidases and the superoxide dismutases can be delivered in combination with one or more of the other listed proteins.

Further embodiments of the invention, including additional proteins, genes and methods for treating and preventing symptoms of xerostomia and xerophthalamia are described below.

B. In Vivo Expression of Genes with Activities Protective Against Xerostomia and Xeropthalmia

Nucleic acids administered according to the compositions and methods of the present invention may encode proteins that have local or systemic effects. Proteins encoded by nucleic acids administered according to the methods of the present invention can be used, for example, to treat or prevent any disorder amenable to treatment or prevention by expression of a therapeutic protein in the blood stream, by secretion of a therapeutic protein to the gastrointestinal tract (e.g,. by secretion of the protein into the saliva), or by expression of the therapeutic protein by the transfected cell, tissue, gland, or organ. The subject can be a mammal such as, for example, a mouse, a rat, a guinea pig, a cat, a dog, a sheep, a goat, a cow, a horse, a koala bear, a non-human primate, or a human; or a non-mammal, such as, for example, a frog, a lizard, a snake, a turtle, a toad, a tortoise, or a salamander.

The methods of the present invention can be used to protect specific tissues in cancer patients against the damaging effects of ionizing radiation and chemotherapeutic drugs, which produce free radicals, superoxide anions, and/or heavy metal cations. In particular, the methods of the present invention can be used to transfer a gene to normal cells at a site remote from the tumor site prior to clinical radiation therapy or chemotherapeutic drug administration to combat cancer. In certain aspects, the methods of the present invention can be used to transfer a gene to normal cells of the oral cavity, oropharynx, esophagus, stomach, small intestine or colon prior to clinical radiation therapy or chemotherapeutic drug administration to combat lung, prostate, bladder, cervical or endometrial cancer, for example.

To obtain high level expression of a cloned gene, such as those cDNAs encoding a suitable therapeutic protein, one typically subclones the gene encoding the protein into an expression vector that contains a strong promoter to direct transcription, a transcription/translation terminator, and if for a nucleic acid encoding a protein, a ribosome binding site for translational initiation. Suitable promoters are well known in the art and described, e.g., in Sambrook et al. (Molecular Cloning, A Laboratory Manual (2nd ed. 1989)) and Ausubel et al (Current Protocols in Molecular Biology (1994)). Eukaryotic expression systems for mammalian cells are well known in the art and are also commercially available. Kits for such expression systems are commercially available.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

Preferably, the nucleic acids used in the methods of the invention comprise a promoter to facilitate expression of the nucleic acid within a salivary gland cell, more preferably a parotid gland cell, even more preferably a submandibular salivary gland cell. Suitable promoters include strong, eukaryotic promoters, for example, cytomegalovirus (CMV), mouse mammary tumor virus (MMTV), Rous sarcoma virus (RSV), and adenovirus promoters. More specifically, suitable promoters include the promoter from the immediate early gene of human CMV (Boshart et al., Cell 41:521 (1985)) and the promoter from the long terminal repeat (LTR) of RSV (Gorman et al., Proc. Natl. Acad. Sci. USA 79:6777 (1982)).

Salivary gland specific promoters may also be used in accordance with the present invention and include, for example, salivary α-amylase promoters and mumps viral gene promoters which are specifically expressed in salivary gland cells. Multiple salivary α-amylase genes, have been identified and characterized in both mice and humans (see, for example, Jones et al., Nucleic Acids Res., 17(16):6613 (1989); Pittet et al., J. Mol. Biol. 182:359 (1985); Hagenbuchle et al., J. Mol. Biol., 185:285 (1985); Schibler et al., Oxf. Surv. Eukaryot. Genes 3:210 (1986); and Sierra et al., Mol. Cell. Biol., 6:4067-(1986) for murine salivary α-amylase genes and promoters; Samuelson et al., Nucleic Acids Res., 16:8261 (1988); Groot et al., Genomics, 5:29 (1989); and Tomita et al., Gene, 76:11 (1989) for human salivary α-amylase genes and their promoters. The promoters of these α-amylase genes direct salivary gland specific expression of their corresponding α-amylase encoding DNAs. These promoters may thus be used in the constructs of the invention to achieve salivary gland-specific expression of a nucleic acid of interest. Sequences which enhance salivary gland specific expression are also well known in the art (see, for example, Robins et al., Genetica 86:191 (1992)).

For eukaryotic expression (e.g., in a salivary gland cell), the construct may comprise at a minimum a eukaryotic promoter operably linked to a nucleic acid operably linked to a polyadenylation sequence. The polyadenylation signal sequence may be selected from any of a variety of polyadenylation signal sequences known in the art, such as, for example, the SV40 early polyadenylation signal sequence. The construct may also include one or more introns, which can increase levels of expression of the nucleic acid of interest, particularly where the nucleic acid of interest is a cDNA (e.g., contains no introns of the naturally-occurring sequence). Any of a variety of introns known in the art may be used.

Other components of the construct may include, for example, a marker (e.g., an antibiotic resistance gene (such as an ampicillin resistance gene)) to aid in selection of cells containing and/or expressing the construct, an origin of replication for stable replication of the construct in a bacterial cell (preferably, a high copy number origin of replication), a nuclear localization signal, or other elements which facilitate production of the nucleic acid construct, the protein encoded thereby, or both.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence and signals required for efficient polyadenylation of the transcript, ribosome binding sites, and translation termination. The nucleic acid sequence may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes.

Some expression systems have markers that provide gene amplification such as thymidine kinase, hygromycin B phosphotransferase, and dihydrofolate reductase. The elements that are typically included in expression vectors also include a replicon that functions in E. coli, a gene encoding antibiotic resistance to permit selection of bacteria that harbor recombinant plasmids or and unique restriction sites in nonessential regions of the plasmid to allow insertion of eukaryotic sequences. The particular antibiotic resistance gene chosen is not critical, any of the many resistance genes known in the art are suitable, for example, the gene for beta-lactamase, which provides resistance to ampicillin, or the kanamyacin resistance gene. The prokaryotic sequences are preferably chosen such that they do not interfere with the replication of the DNA in eukaryotic cells.

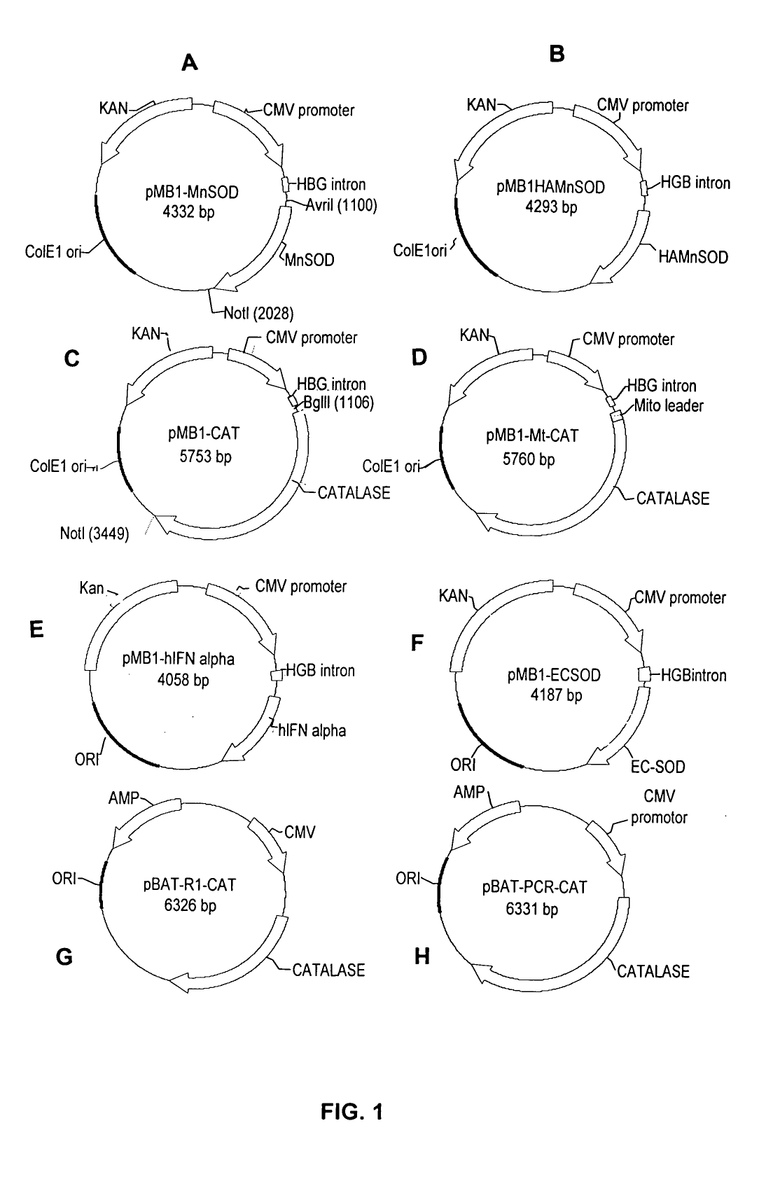

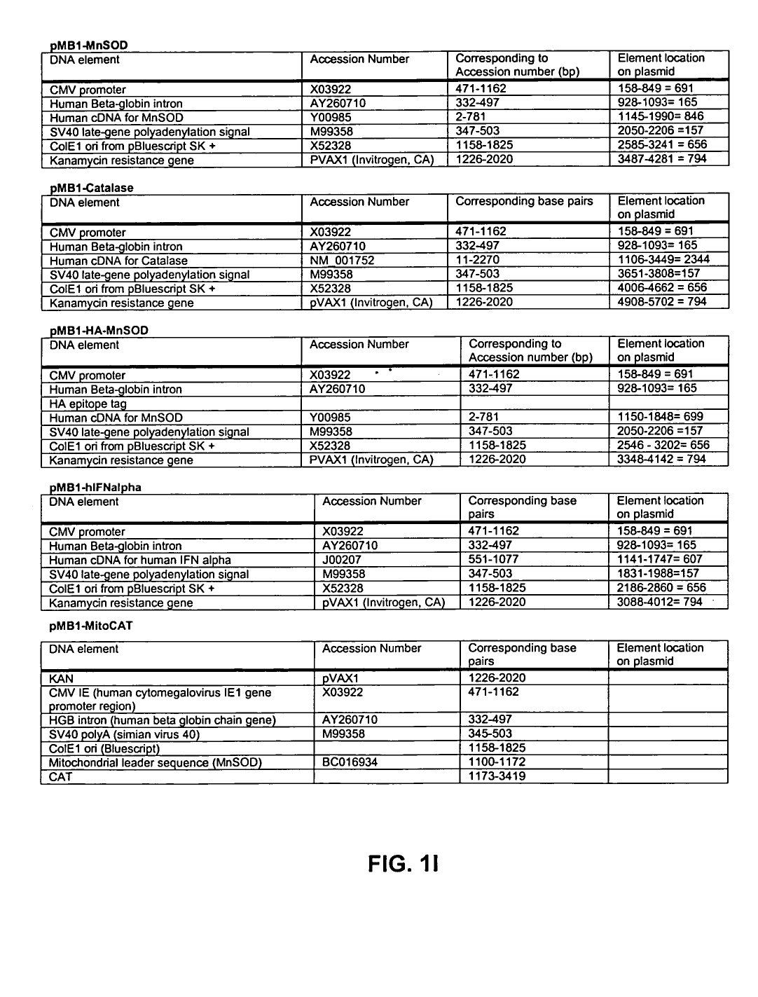

Preferred vectors for protecting subjects from symptoms of xerostomia or xeropthalmia, or ameliorating symptoms of xerostomia or xerophthalmia, are illustrated in FIG. 1A-H. The nucleic acid sequences for these vectors are provided as SEQ IDs Nos. 1-8. One skilled in the art will recognize that many changes may be made to the nucleic acid sequences of these vectors without substantially altering the functionality of any genes encoded by the nucleic acids. Preferably, any changes made to the nucleic acid sequences will increase or retain the activity or stability of the genes encoded by the plasmids, particular those genes which encode the eukaryotic proteins which impart the vectors with their anti-xerostomic or anti-xerophthalmic activities.

C. Polyionic Organic Acids

In certain aspects, the present invention provides methods for treating or protecting a tissue from symptoms of xerostomia or xerophthalmia by administering to a subject a composition comprising a polyionic organic acid. Preferably, the polyionic organic acid is a dye. In certain aspects, the dyes include, but are not limited to, suramin, Evans Blue, Congo Red, ponceau S, Congo corinth, Sirius red F3B, ponceau 6R, amido black 10B, biebrich scarlet and aurintricarboxylic acid (“ATA”). Preferably, the dye absorbs in the visible light spectrum. A “polyionic organic acid” (POD) as used herein, is preferably a polyprotic polyaromatic organic compound wherein the compound contains at least two aromatic components (see, U.S. Provisional Application No. 60/402,811 filed Aug. 12, 2002, and PCT Publication PCT/US03/25419, both of which are incorporated herein by reference). “Polyionic” compounds refer to compounds comprising one or more ionizable units, either in the protonated form or as the conjugate salt. In certain embodiments, the POD is associated or complexed with a transition metal enhancer, such as zinc.

Dyes which are useful in the present invention include, but are not limited to, an acid dye, a disperse dye, a direct dye and a reactive dye. In a preferred embodiment, an acid dye is used. Suitable acid dyes include, but are not limited to, direct red dye, direct blue dye, acid black dye, an acid blue dye, an acid orange dye, an acid red dye, an acid violet dye, and an acid yellow dye. In certain other preferred embodiments, suitable acid dyes include, but are not limited to, Evans Blue, Congo Red, ponceau S, Congo corinth, Sirius red F3B, ponceau 6R, amido black 10B, biebrich scarlet and aurintricarboxylic acid. In yet another preferred embodiment, a direct dye is used. Preferred direct dyes include direct red, direct blue, direct yellow and direct green. More preferably, direct blue 15 (Light Blue), direct red 28 (Congo Red) and direct blue 53 (Evans Blue) are used. Preferably, the dye absorbs in the visible light spectrum, between about 400 nm to 700 nm.

In another preferred embodiment, the polyionic organic acid and nucleic acid composition is an aqueous solution having a physiological acceptable pH.

In a preferred embodiment, the polyionic organic acid is comprised of a complex formed between suriman and a transitional metal enhancer, for example, a divalent metal halide such as zinc chloride. Surprisingly, the addition of a divalent metal to a formulation with suriman and DNA results in a synergistic increase in gene expression. In certain aspects, the in vitro concentrations of PODS used herein are about 10 to about 0.0001 mg/mL. Preferably, the concentration of PODS used herein is about 1 to about 0.001 mg/mL. More preferably, the concentration of PODS used herein is about 0.1 to about 0.01 mg/mL.

D. Transition Metal Enhancers

In certain other embodiments, the nucleic acid or protein compositions of the present invention for treating or preventing symptoms of xerostomia or xerophthalmia further comprise an ionizable or ionized transition metal enhancer, including, but not limited to, a complex, an adduct, a cluster or a salt of an element including, but not limited to, a d-block element, a first row f-block element, aluminum and gallium. In a preferred embodiment, the ionizable or ionized transition metal enhancer is a complex, an adduct, a cluster or a salt of an element including, but not limited to, zinc, nickel, cobalt, copper, aluminum and gallium. More preferably, the ionizable or ionized transition metal enhancer includes, but is not limited to, zinc sulfate, zinc acetate, nickel sulfate, nickel acetate, cobalt sulfate, cobalt acetate, copper sulfate and copper acetate. In another preferred embodiment, the ionizable or ionized transition metal enhancer includes, but is not limited to, zinc acetate or zinc sulfate. In other embodiments, the ionizable or ionized transition metal enhancer is a metal halide including, but not limited to, zinc halide, nickel halide, cobalt halide, copper halide, aluminum halide and gallium halide. In certain preferred embodiments, the ionizable or ionized transition metal enhancer includes, but is not limited to, ZnCl₂, NiCl₂, CoCl₂, CuCl₂, AlCl₂ and GaCl₂. In a related embodiment, the transition metal enhancers or PODS or both or administered to a subject as part of a treatment regimen which includes the nucleic acids or proteins of the invention.

E. Administration of Nucleic Acids and other Compounds

According to the present invention, the nucleic acid can be administered according to any means known in the art. For example, the nucleic acids of the present invention can be administered to a subject using electroporation. Suitable methods of administration of the nucleic acid to the cells, tissues, glands, or organs include, for example, cannulation or injection of the nucleic acid into the cells, tissues, glands, or organs using a syringe, cannula, catheter, or shunt. The type of syringe used is not a critical part of the invention. One of skill in the art will appreciate that multiple types of syringes may be used to administer nucleic acids according to the present invention. Suitable types of syringes include, for example, an aspirating syringe, a removable needle syringe, a modified microliter syringe, a microliter syringe, a gastight syringe, a sample lock syringe, or a threaded plunger syringe.

The present invention also relates to a process for administering a nucleic acid to a secretory gland cell. Suitable secretory gland cells include, but are not limited to, a salivary gland cell, a pancreatic cell, a mammary gland cell, a thyroid cell, a thymus cell, a pituitary gland cell, and a liver cell. It is particularly preferred that the secretory gland cell is a salivary gland cell.

The nucleic acid composition can be delivered to the secretory gland cell or tissue of the mammal by intraductal delivery or direct administration. A preferred method of delivery is by electroporation administration. Preferably, the process for administering a nucleic acid composition to a secretory gland cell includes an electroporation administration that comprises contacting the salivary gland with an electrode comprising at least two needles and pulsing the salivary gland.

Delivery of the nucleic acid may be via gravity or an assisted delivery system. Suitable assisted delivery systems include controlled release pumps, time release pumps, osmotic pumps, and infusion pumps. The particular delivery system or device is not a critical aspect of the invention. One of skill in the art will appreciate that multiple types of assisted delivery systems may be used to deliver nucleic acids according to the methods of the present invention. Suitable delivery systems and devices are described, for example, in U.S. Pat. Nos. 5,492,534, 5,562,654, 5,637,095, 5,672,167, and 5,755,691. One of skill in the art will also appreciate that the infusion rate for delivery of the nucleic acid may be varied. Suitable infusion rates may be from about 0.005 mmin to about 1 ml/minute, preferably from about 0.01 ml/min to about 0.8 mmin., more preferably from about 0.025 mmin. to about 0.6 ml/mn. It is particularly preferred that the infusion rate is about 0.05 ml/min.

In accordance with the present invention, the nucleic acid is administered with a formulant that enhances transfection efficiency. Suitable formulants include, for example, divalent transition metals, polyionic compounds, and peptides. Suitable divalent transition metal compounds include, for example, zinc halide, zinc oxide, zinc acetate, zinc selenide, zinc telluride, and zinc sulfate. Preferred suitable divalent transition metal compounds include, for example, ZnCl₂, CuCl₂, CoCl₂, NiCl₂, and MgSO₄ (Shiokawa et al., Biochem J. 326:675 (1997) and Torriglia et al., Biochimie 79:435 (1997)). Other suitable divalent transition metals are described in U.S. patent application Ser. No. 09/487,089, filed Jan. 19, 2000, U.S. patent application Ser. No. 09/766,320, filed Jan. 18, 2001, and International Publication WO 01/52903, filed Jan. 19, 2001. Suitable polyanionic compounds include, for example, poly-L-glutamate. Suitable peptides include, for example, ID2 and peptides based on it such as, for example ID2-2, ID2-3, ID2-4 (Sperinde et al., J. Gene Med. 3:101 (2001)). Other suitable formulants include, for example, polyvinyl alcohol and nuclease inhibitors (Glasspool-Malone, et al. Mol. Ther. 2(2): 140 (2000)), sodium citrate, and G-actin (Shiokawa et al. (1997), supra).

The compositions of the present invention can also be localized in the secretory gland cell of the mammal by a process of delivering the composition to the mammal by direct administration or intraductal delivery. One process for localizing a nucleic acid composition in a secretory gland is by electroporation administration, for example, contacting the salivary gland with an electrode comprising at least two needles and pulsing the salivary gland. Methods relating to electroporation are described, for example, in U.S. patent application Ser. No. 10/126,315, filed Apr. 19, 2002.

Retroductal Gene Introduction

The present invention also provides methods for protecting or treating a tissue or cell from a condition that elicits xerostomia or xerophthalmia by retroductally introducing into the lumen of a salivary or lacrimal gland duct of a subject a composition, preferably a nucleic encoding a protein, which is capable of neutralizing a harmful species. Retroductal delivery of nucleic acids is particularly effective for treating xerostomia or xerophthalmia according to the methods of the invention.

For example, in a preferred embodiment, a composition comprising a nucleic acid encoding one or more of the proteins described herein as capable of ameliorating a symptom of xerostomia or xerophthalmia, or providing protection against either condition, is retroductally introduced into the lumen of a salivary or lacrimal gland duct. The nucleic acid may be in a vector, or may be “naked” as described in, e.g., Ulmer et al. (1993) Science 259:1745-1749 and reviewed by Cohen (1993) Science 259:1691-1692. The composition may be introduced alone or with an adjuvant. In some embodiments of the present invention, the adjuvant is administered at the same time as the composition. In other embodiments of the present invention, the adjuvant is administered after the composition, e.g., 6, 12, 18, 24, 36, 48, 60, or 72 hours after administration of the composition.

Suitable methods of retroductal introduction of the composition to the salivary or lacrimal gland duct include, for example, cannulation or injection of the composition into the salivary gland duct using a syringe, cannula, catheter, or shunt. The type of syringe, cannula, catheter, or shunt used is not a critical part of the invention. One of skill in the art will appreciate that multiple types of syringes, cannulas, catheters, or shunts may be used to administer compositions according to the methods of the present invention.

Retroductal delivery of the composition using the methods of the present invention may be via gravity, syringe or an assisted delivery system. Suitable assisted delivery systems are described herein.

Application of gene transfer would typically occur one to seven days prior to radiotherapy, to allow time for the gene to be taken up and expressed in the target cells. After instillation, the catheter is left in place (seconds to minutes) to ensure that the gene vector is able to contact the target cells. Proteins or chemicals may be applied immediately prior to radiotherapy. In this case, it may be advantageous to hold the material in place during and immediately following radiotherapy. Sialography has revealed that fluid is cleared quickly from salivary glands, and much of the material passes back through the catheter if allowed. Leaving the catheter in place and the line closed ensures that the fluid remains in the gland. In the case of proteins or chemicals, it may also be advantageous to apply the mater (once or multiple ties) after radiotherapy to help clear remaining harmful compounds.

In addition to genes that encode for antioxidant proteins, radioprotective chemicals may also be administered through the ducts to protect salivary and lacrimal gland tissues. Candidate chemicals for this purpose include cytoprotectants that are currently approved for clinical use (amifostine, dexrazoxane, and mesna) and those that are currently under preclinical development (e.g., phosphorothioate analogs, thiolamines, selenazolidien prodrugs, and antioxidant proteins-MnSOD, Cu/ZnSOD, and the like). In addition, PODS such as suramin, Congo Red, and Evans Blue may also serve as cytoprotectants. These radioprotective chemicals can be administered alone (either before or during radiation therapy) or in conjunction with an antioxidant gene. When co-administered with an antioxidant gene, these radioprotective chemicals can serve dual roles as transfection enhancers and as cytoprotectants.

Frequency of administration of the prophylactic or therapeutic compositions described herein, as well as dosage, will vary from individual to individual, and may be readily established using standard techniques. Often between 1 and 10 doses may be administered over a 52-week period. Typically, 6 doses are administered, at intervals of 1 month, more typically, 2-3 doses are administered every 2-3 months. Booster vaccinations may be given periodically thereafter. Alternate protocols may be appropriate for individual patients and particular diseases and disorders. A suitable dose is an amount of a compound that, when administered as described above, is capable of promoting, e.g., a protective response, and is at least 10-50% above the basal (i.e., untreated) level. Typically, the amount of the nucleic acid encoding a polypeptide present in a dose ranges from about 1 μg to 5 mg, preferably 100 μg to 5 mg, and most preferably 5 μg to 300 μg per kg of host. Suitable dose sizes will vary with the size of the patient, but will typically range from about 0.01 ml to about 10 ml, more typically from about 0.025 to about 7.5 ml, most typically from about 0.05 to about 5 ml. Those of skill in the art will appreciate that the dose size may be adjusted based on the particular patient or the particular disease or disorder being treated.

In general, an appropriate dosage and treatment regimen provides the active compound(s) in an amount sufficient to provide therapeutic and/or prophylactic benefit. Such a response can be monitored by establishing an improved clinical outcome in treated patients as compared to non-treated patients.

### EXAMPLES

The following examples are offered to illustrate, but not to limit the claimed invention.

### Example 1

### Expression of Recombinant Catalase in Rat Submandibular Salivary Glands

Materials and Methods: To demonstrate that recombinant catalase could be expressed efficiently in transfected salivary glands, submandibular glands of SD rats were treated with pBATR1-CAT, pBAT-PCR-CAT, and a control vector, pSEAP (ClonTech Laboratories, Saint-Quentin, France), which encodes secreted alkaline phosphatase under control of the early adenovirus SV40 promoter. A fourth set of rats was left untreated, i.e., “naive.”

Intraductal administration of a DNA construct to the rat submandibular glands. Male Sprague-Dawley rats (260-280 g, Harlan Laboratories, San Diego, Calif.) were fasted the night prior to treatment. After intramuscular administration of a mixture of ketamine (30 mg/kg b.wt.), xylazine (6.0 mg/kg b.wt.), and acepromazine (1.0 mg/kg b.wt), both the right and left Wharton's ducts were cannulated with fine polyethylene tubing (i.d. 0.011″) and cemented in place with a quick drying adhesive. Atropine (0.5 mg/kg b.wt.) was then administered subcutaneously and, after ten minutes, the plasmid DNA solution was administered through the polyurethane tubing. The plasmid DNA solution, 50 microliters, contained plasmid DNA (175 micrograms, encoding the gene of interest), suramin (a polyionic organic acid; 40 mg/ml), zinc chloride (5 mM) and saline (0.9%). The tubing was removed 10 minutes later. When indicated, dexamethasone (1.5 mg/kg b.wt) was administered via intramuscular injection immediately prior to DNA administration.

Collection of salivary gland tissue and blood samples to assay for transgene expression. At 48 hours post-DNA administration, the rats were anesthetized by intraperitoneal injection of sodium pentobarbital (50 mg/kg b.wt.). Plasma was collected from heparinized whole blood by cardiac puncture and stored at −80° C. until assay. The right and left submandibular glands were surgically excised. All research conducted using these experimental animals adhered to the “Principals of Laboratory Animal Care” (NIH publication #85-23, revised in 1985).

RT-PCR assay of gene expression. The harvested gland tissues were lysed and the RNA was extracted according to the instructions provided with the RNeasy Kit (Qiagen). The RNA was treated with DNase to remove plasmid and genomic DNA contaminations. One-Step RT-PCR reagents (Invitrogen) and a primer specific to the 5′UTR of the pBAT/pMB1 plasmid backbone (5′GGAGACGCCATCCACGCTGTT; SEQ ID NO: 9) and a 3′ oligo specific for the human catalase gene (5′CGATACCGTCGACCTCGAGAGA; SEQ ID NO:11) were used to detect plasmid-specific catalase transcripts. The PCR oligomers were designed to flank the human globin intron so that any contamination from plasmid DNA would be detected. A similar strategy was used in experiments described below to detect the MnSOD genes, except that the 3′ oligo was MnSOD specific (5′GCGCGTTGATGTGAGGTTCC; SEQ ID NO:10). The same primer specific for the 5′UTR of the plasmid vector was used in conjunction with the MnSOD specific primer to detect the presence of MnSOD specific transcripts. Thermal cycling conditions were 30 min at 50° C. for RT followed by 5 min at 95° C. and 40 cycles of 30 s at 95° C., 30 s at 58° C. and 45 s at 72° C. The samples were then run on an agarose gel. Amplicons were sequenced to confirm that they were derived from the targeted gene.

Measurement of catalase activity in submandibulargland tissue. Harvested tissue was minced with surgical scissors and transferred to a tube. Sodium phosphate lysis Buffer (50 mM NaHPO4, pH 7.0, 1 mM EDTA) at 15× per gram of tissue was added and the mixture homogenized at high speed for 15 minutes. The homogenate was centrifuged at 12K RPM for 20 minutes at 10° C., and the supernatant was collected and stored at −80° C. until analysis.

Catalase activity in rat SMG samples was determined using Cayman Catalase Assay Kit (Cayman Chemical Company, Ann Arbor, Mich.), following manufacturer's instructions. In this assay, catalase in the sample reacts with methanol and hydrogen peroxide to produce formaldehyde, which in turn reacts with a chromagen to allow spectrophotometric measurement. The standard curve for this assay is generated from a formaldehyde standard which reacts with the chromagen. A simple calculation involving the sample dilution and the duration of the assay converts the formaldehyde concentration generated by the sample into catalase activity.

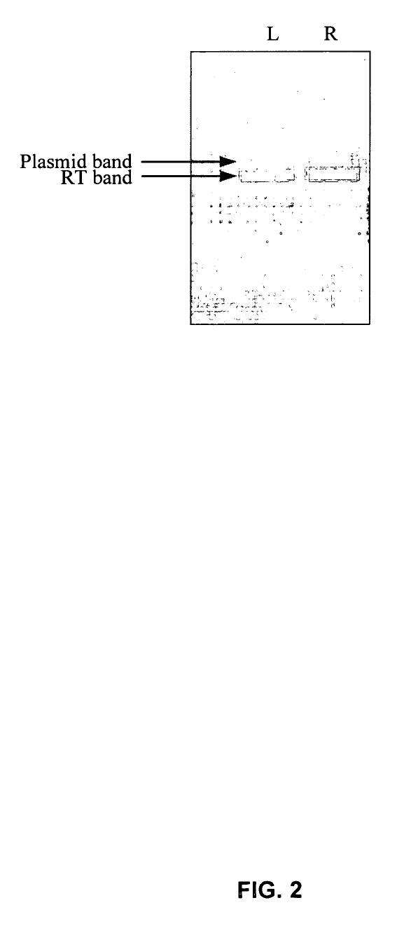

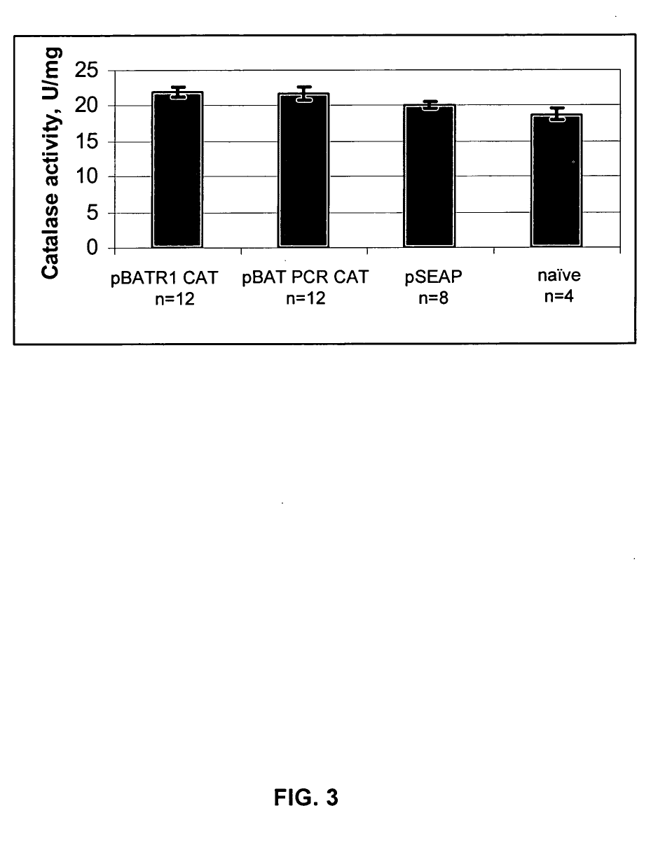

Results: The results in FIG. 2 show significant transcription of catalase in submandibular gland tissue from a rat 48 hours after transfection of the rat's submandibular glands with pMB 1-CAT, using the methods described above. FIG. 3 shows that the methods and compositions described above for transfection of rat submandibular salivary glands with a vector encoding catalase lead to significant increases in catalase activity in the transfected tissue. Specifically, transfection with either pBATR1-CAT or pBAT-PCR-CAT resulted in greater catalase activity relative to (1) a control vector encoding secreted alkaline phosphatase (pSEAP) or (2) untreated rats. In both the pSEAP-treated rats and the untreated rats, the only observed catalase activity is the catalase activity endogenous to rat submandibular salivary glands.

### Example 2

### Expression of Recombinant MnSOD in Rat Submandibular Salivary Glands

Materials and Methods: To demonstrate that a superoxide dismutase enzyme could be expressed efficiently in salivary glands, the protocols described in Example 1 were followed to transfect rats with vectors encoding MnSOD, e.g., pMB1-MnSOD and pMB1-HA-MnSOD. 48 hours after treatment, submandibular salivary gland tissue samples were collected. To detect transcription, RNA was extracted from the samples infected with pMnSOD and assayed by RT-PCR with MnSOD-specific primers, as described in Example 1.

Staining of Frozen Sections of Salivary Gland Tissue.

Frozen tissue was cut into 6 micrometer sections, fixed, and stained with antibodies according to standard protocols. The primary antibody used to detect MnSOD expression was a rabbit polyclonal antibody against MnSOD (USBiologicals Cat#S8060-10A, 1:100 in 1% BSA in PBS). The primary antibody used to detect HA-MnSOD expression was a mouse monoclonal antibody against 12 amino acids (YPYDVPDYA) in the influenza hemaglutinin epitope (Covance Cat#MMS-101R, 1:500 in 1% BSA in PBS). Peroxidase activity was used to visualize sites of antibody binding (i.e., sites of superoxide dismutase expression) in the presence of a methyl green counterstain.

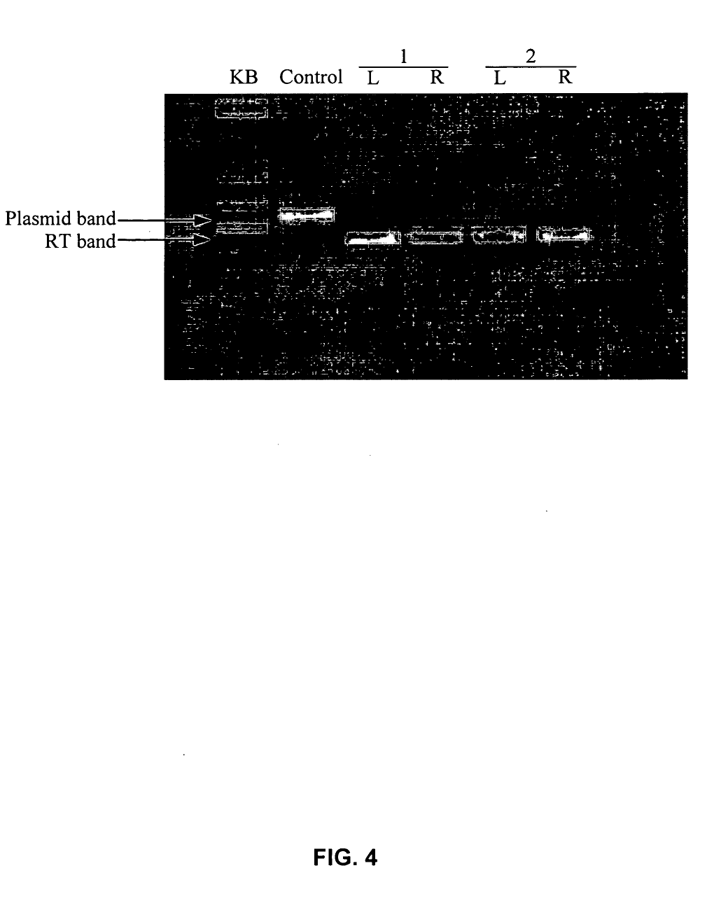

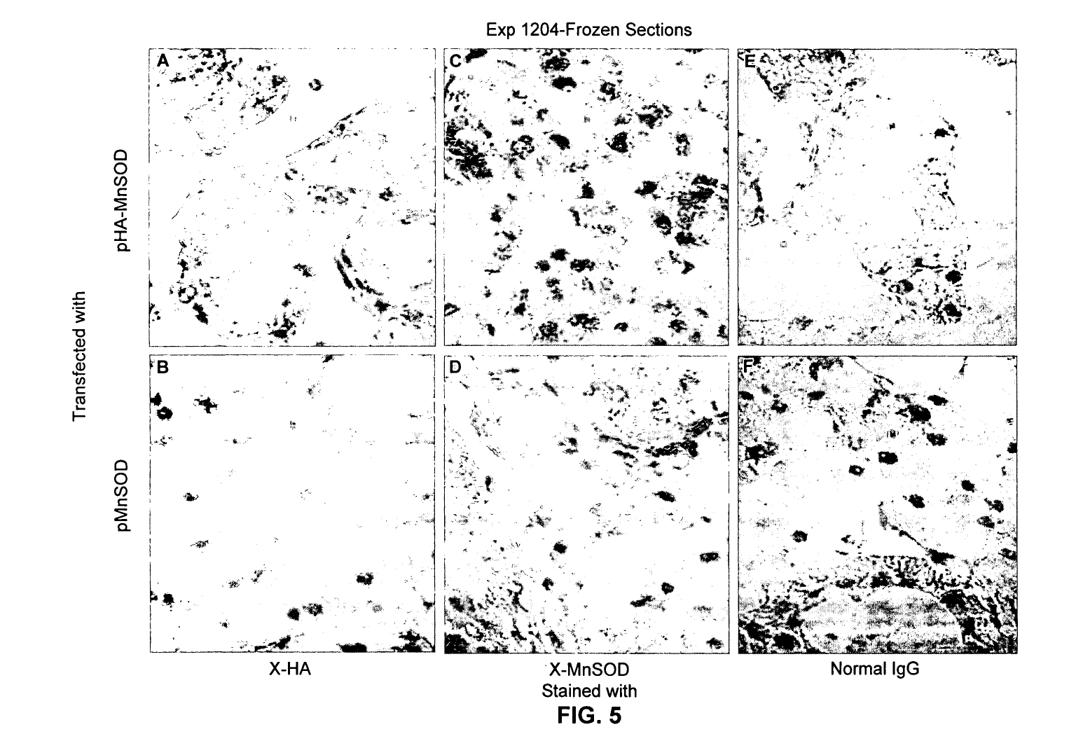

Results: The results of an RT-PCR assay on two rats transfected with pMB1-MnSOD appear in FIG. 4 which shows that significant levels of MnSOD transcription were observed in glands taken from both rats. FIG. 5A-F shows the results of experiments designed to detect the expression of recombinant manganese superoxide dismutase proteins in pMB1-MnSOD and pMB 1-HA-MnSOD transfected submandibular glands of rats. Comparing panel A to panels B, E or F in FIG. 5 shows that HA-MnSOD protein is expressed at significantly detectable levels in transfected salivary gland tissue (the anti HA antibody doesn't bind to MnSOD lacking the hemaglutinin epitope.

Likewise, comparing panels C and D to panels E and F shows that MnSOD is expressed at significantly detectable levels in transfected salivary gland tissue. Note that staining in transfected glands is not limited to the salivary ducts, where some endogenous MNSOD is found. Importantly, in transfected glands, staining is visible in both the acini and ducts of salivary gland tissue.

### Example 3

### MnSOD Prevents Irradiation Damage to Salivary Glands

Recombinant human MnSOD is effective in reducing tissue damage following exposure to whole-body or localized irradiation in laboratory animals. Recombinant human MnSOD (enzyme, 50 mg/kg) injected interperitoneally 90 minutes prior to irradiation protects parotid glands from radiation-induced hyposalivation. MnSOD injection is effective against radiation-induced esophagitis and mucocitis.

Animals, including rats and humans, may be assayed for xerostomia by measuring physiological variables such as body weight and salivary gland function. The lack of saliva in animals affected with xerostomia often leads to tooth decay and other painful oral conditions which prevent the comfortable ingestion of food. In irradiated animals, water and food intake significantly lowers following irradiation. White blood cell counts are also reduced (myelosuppression).

Animals which have been irradiated or are about to be irradiated will benefit from the transfection of their salivary glands with recombinant vectors expressing the proteins described herein. For example, prior to irradiation, a vector encoding MnSOD may be retroductally delivered to the submandibular salivary glands of a group of test animals. A control group of animals will either not receive an injection or receive a vector which includes a control protein, such as alkaline phosphatase. Both groups of animals will be irradiated and tested for salivary gland function.

For example, if the animal is a rat, the salivary glands may be tested by measuring the volume of saliva secreted following intraperitoneal pilocarpine-HCL stimulation, as described by Nagler et al. (Rad. Research, 136:392-96 (1993)). Salivary gland function in animals pre-treated with the vector encoding MnSOD will be significantly closer to normal than salivary gland function in the untreated or control animals. Similar tests, as well as subjective tests such as interviews relating to discomfort and dryness of the mouth, may be used to measure the benefits to humans who have been pre-treated prior to irradiation.

The tests described above may be performed on animals who have been diagnosed as suffering from xerostomia, either because the animals have been irradiated or because the animals are suffering from a disease such as Sjögren's disease. For example, irradiated animals which exhibit diminished salivary gland function or other symptoms of xerostomia may receive retroductal delivery of a vector expressing MNSOD or one of the other irradiation-protective proteins described herein, as a form of post-irradiation therapy. The effects of the post-irradiation delivery may be measured using standard assays such as the salivary gland function assay described by Nagle et al. (1993).

Analogous experiments may be carried to show the beneficial effects of similar treatments of lacrimal glands, prior to and/or following irradiation.

Overexpression of MnSOD does not have any side effects. Targeted delivery of MnSOD to affected cells reduces the frequency of re-administration. The salivary glands, for example, provide such a targeted route for treatment of xerostomia. Moreover, targeted delivery is safer than other delivery alternatives.

### Example 4

### Treatment of Siogren's Syndrome With Interferon Alpha

This Example illustrates the treatment of Sjögren's syndrome with retroductal salivary gland interferon alpha (IFN-α).

Sjögren's syndrome is an autoimmune disorder characterized by lymphocytic infiltration of multiple organs (including salivary glands). Symptoms of Sjögren's syndrome include xerostomia (dry mouth) and xerophthalmia (dry eyes). Oral symptoms of xerostomia include: dry mouth, difficulty eating and speaking, increase in the prevalence of dental caries, recurrent yeast infection, retention of food and bacteria, and swelling and tenderness of the major salivary glands. There are approximately 500,000-2,000,000 cases of Sjögren's syndrome in the United States. Ninety percent of all patients are women. The onset of disease is during the fourth or fifth decade of life.

Currently, remedies for Sjögren's syndrome only treat the disease symptoms. However, most treatments do not effectively palliate symptoms. Pilocarpine tablets treat Sjögren's syndrome symptoms. Other treatments include artificial saliva or tears, mouthwashes, and sodium fluoride. Drugs currently under development include active salivary gland gene transfer with IL-4 and IL-10, daily low dose oral interferon alpha to increase salivary and lacrimal flow and reduce salivary gland inflammation, and weekly i.m. interferon alpha injections.

Intramuscular injections of recombinant IFN-α, given three times at a dose of 3×10⁶ MU per week had a response rate of 70% (salivary function) and 50% (lacrimal function). The intramuscular IFN-α treatment increased salivary and lacrimal function by 67% and 61%, respectively. No patients discontinued the therapy. In contrast to the intramuscular IFN-α, clinical studies of patients taking three intramuscular IFN-α lozenges a day had mixed results, and a high placebo effect was observed.

A non-viral delivery of IFN-α to salivary gland six times per year generates continuously efficacious levels of IFN-intramuscular IFN-α. The cell-based therapy that targets afflicted cells has numerous advantages: 1) it reduces the frequency of re-administration, 2) it is safer than other delivery alternatives, and 2) it provides a route for targeted cell therapy.

### Example 5

### Expression of IFN-α in Rat Submandibular Salivary Glands

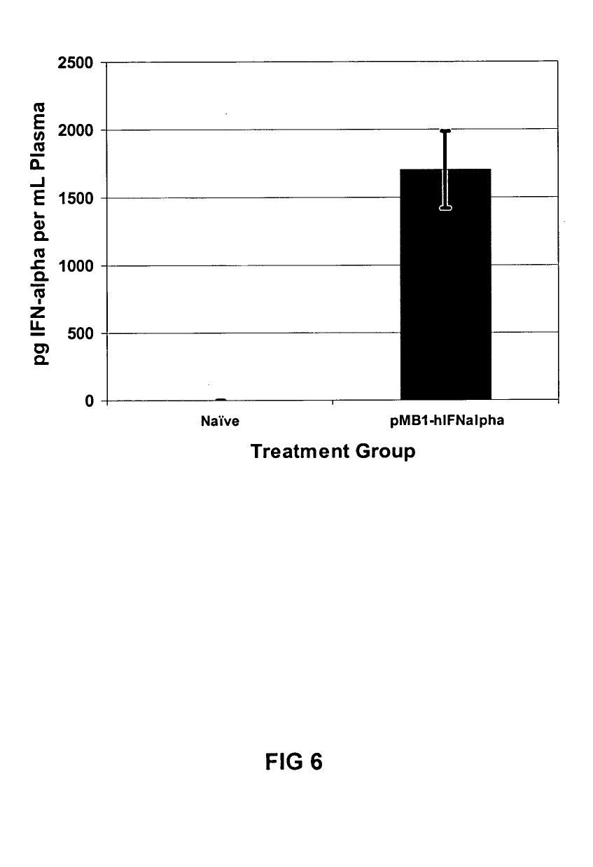

To demonstrate that gene transfer to the salivary glands can be used to generate therapeutic anti-inflammatory cytokines, 50 microliters of a solution containing plasmid DNA encoding for human alpha interferon(pMB1-hIFNalpha,175 micrograms), saline (0.9%), suramin (40 mg/mL), and zinc chloride (5 mM) was administered to the right and left submandibular glands of adult, male Sprague-Dawley rats according to the protocol descrbed in Example 1. At 48 hours post DNA administration, plasma was collected from the animals and assayed for human alpha interferon concentration using a commercially available alpha interferon ELISA kit (PBL Biomedical Laboratories, New Brunswick, N.J.). FIG. 6 shows the average alpha interferon concentration found in the plasma of 4 treated rats, SEM. As a control, plasma was collected from untreated, naive rats (n=3) and assayed for alpha interferon concentration. The results from this study demonstrate that gene transfer to salivary glands can be used to produce human alpha interferon. Since the human alpha interferon is secreted into the systemic circulation, gene transfer to the salivary glands could also be used to deliver anti-inflammatory agents to non-salivary gland tissues (i.e. other glands, liver, pancreas, etc, etc). The expression pattern thus allows salivary gland transfection by IFN-α to be used as a tool for the treatment of a variety of conditions including, but not limited to, Sjögren's syndrome, inflammatory conditions, and other immune disorders which may not necessarily include salivary gland dysfunction as an indication.

Although the foregoing invention has been described in detail by way of illustration and example for purposes of clarity and understanding, it will be readily apparent to one of ordinary skill that, in light of the teachings herein, certain changes and modifications may be made to the claimed invention without departing from its spirit or scope. All publications and patent applications cited in this specification are herein incorporated by reference as if each individual publication or patent application were specifically and individually indicated to be incorporated by reference in their entirety for all purposes.

1. A method for attenuating increases in concentrations of radiation-induced free radicals, superoxide anions or heavy metal cations in a mammalian cell, said method comprising:
contacting said cell with one or more nucleic acids encoding one or more proteins, wherein said one or more proteins are expressed in said cell, and wherein said one or more proteins neutralizes or eliminates a portion of said free radicals, superoxide anions or heavy metal cations in said cell.

2. The method of claim 1, wherein said one more proteins are selected from the group consisting of a metallothionein, superoxide dismutase, catalase, glutathione peroxidase (GPx)-4, or gamma glutamyl transpeptidase.

3. The method of claim 2, wherein said one or more proteins is a metallothionein.

4. The method of claim 2, wherein said one or more proteins is a superoxide dismutase.

5. The method of claim 2, wherein said one or more proteins is a catalase.

6. The method of claim 2, wherein said one or more proteins is a gamma glutamyl transpeptidase.

7. The method of claim 1, wherein said one or more proteins is glutathione peroxidase.

8. The method of claim 4, wherein said superoxide dismutase is selected from the group consisting of a copper-zinc superoxide dismutase, manganous superoxide dismutase and iron superoxide dismutase.

9. The method of claim 1, wherein said cell is a human cell.

10. The method of claims 9, wherein said contacting occurs in vivo prior to irradiation of the cell with X-rays.

11. The method of claims 9, wherein said contacting occurs in vivo following irradiation of the cell with X-rays.

12. The method of claim 10, wherein said cell is a salivary gland cell and wherein said contacting is repeated on a plurality of cells, and wherein said expression of said one or more proteins is sufficient to ameliorate a symptom of xerostomia in the subject.

13. The method of claim 11, wherein said cell is a salivary gland cell and wherein said contacting is repeated on a plurality of cells, and wherein said expression of said one ore more proteins is sufficient to ameliorate a symptom of xerostomia in the subject.

14. The method of claim 10, wherein said cell is a lacrimal gland cell and wherein said contacting is repeated on a plurality of cells, and wherein said expression of said one ore more proteins is sufficient to ameliorate a symptom of xerophthalmia in the mammal.

15. The method of claim 11, wherein said cell is a lacrimal gland cell and wherein said contacting is repeated on a plurality of cells, and wherein said expression of said on ore more proteins is sufficient to ameliorate a symptom of xerophthalmia in the mammal.

16. The method of claim 1, wherein said nucleic acid is an expression vector.

17. The method of claim 16, wherein said expression vector is selected from the group consisting of SEQ ID 1 (pMB1-MnSOD), SEQ ID 2 (pMB1-HAMnSOD), SEQ ID 3 (pMB 1-CAT), SEQ ID 4 (pMB 1-Mt-CAT), SEQ ID 5 (pMb1-hIFN-alpha), SEQ ID 6 (pMB1-EcSOD), SEQ ID 7 (pBAT-R1-CAT) and SEQ ID 8 (pBAT-PCR-CAT).

18. The method of claim 1, wherein said cell is a salivary gland cell and wherein said nucleic acid is directly or intraductally delivered to said cell in vivo, and wherein said contacting is repeated with a plurality of salivary gland cells, and wherein said protein expression is sufficient to ameliorate a symptom of xerostomia in said mammal.

19. The method of claim 1, wherein said cell is a lacrimal gland cell and wherein said nucleic acid is directly or intraductally delivered to said cell in vivo, and wherein said contacting is repeated with a plurality of lacrimal gland cells, and wherein said protein expression is sufficient to ameliorate a symptom of xerophthalmia in said mammal.

20. The method of claim 1, wherein said method further comprises contacting said cell with a composition comprising a polyionic organic acid.

21. The method of claim 20, wherein said composition further comprises a transition metal enhancer.

22. The method of claim 21, wherein said transition metal is zinc.

23. A plurality of recombinant salivary gland cells in a mammal, wherein said cells comprise one or more vectors for expressing a protein which neutralizes or eliminates a portion of free radicals, superoxide anions or heavy metal cations in said cells, and wherein said expression is sufficient to ameliorate a symptom of xerostomia in said mammal.

24. A method for ameliorating a symptom of xerostomia in a mammal, said method comprising:
contacting a salivary gland cell of said mammal with a composition comprising a protein selected from the group consisting of IFN-alpha, IL-10, sTNFR, TGF-β, IL-4 and VIP, anti-TNF antibody, IL1-RA, other antibodies to proinflammatory cytokines, soluble gp39, soluble CD40, aquaporin-1 and aquaporin-5.

25. The method of claim 24, wherein said protein is IFN-alpha.

26. The method of claim 24 or 25, wherein said protein is secreted in a body fluid.

27. A method for ameliorating a symptom of xerostomia in a mammal, said method comprising:
contacting a salivary gland cell of said mammal with a composition comprising a nucleic acid, wherein said nucleic acid encodes an IFN-alpha protein, and wherein said encoded IFN-alpha protein is expressed in said cell at a level sufficient to ameliorate a symptom of xerostomia in said mammal.

28. The method of claim 27, wherein said mammal is a human and wherein said symptom is associated with a condition selected from the group consisting of:
an autoimmune disorder, Sjogren's syndrome, graft-versus-host disease, systemic lupus erythematosis, rheumatoid arthritis, HIV-1 infection, ageing, autonomic dysfunction, conditions affecting the CNS, psychogenic disorder, trauma, hepatitis C, cancer and decrease in mastication.

29. The method of claim 28, wherein at least a portion of said expressed IFN-alpha protein is secreted from said salivary gland cell in a body fluid.

30. The method of claim 29, wherein said body fluid is saliva.

31. The method of claim 29, wherein said body fluid is blood plasma.

32. The method of claim 28, further comprising contacting the salivary gland of said mammal with a composition comprising a polyionic organic acid.

33. The method of claim 28, wherein said composition further comprises a transition metal enhancer.

34. An isolated nucleic acid selected from the group consisting of SEQ ID NOs 1-8.
